(19) United States
(12) Patent Application Publication
(10) Pub. No.: US 2024/0315606 A1
(43) Pub. Date: Sep. 26, 2024

(54) METHOD OF IMPROVED SURGICAL CARE WITH REAL-TIME DEVICES

(71) Applicant: Know Labs, Inc., Seattle, WA (US)

(72) Inventor: John CRONIN, Williston, VT (US)

(21) Appl. No.: 18/187,324

(22) Filed: Mar. 21, 2023

(51) Int. Cl. A61B 5/145; A61B 5/00; A61B 5/0205; A61B 5/07

(52) U.S. Cl. CPC A61B 5/14532; A61B 5/0205; A61B 5/0507; A61B 5/07; A61B 5/14542; A61B 5/4818; A61B 5/7246; A61B 5/7275; G16H 50/20; G16H 50/30; A61B 5/021

(57) ABSTRACT

A method of improved surgical care with real-time devices in which a real-time glucose monitoring device and real-time non-glucose devices measure a patient's physiological parameters during surgery. The physiological parameters are analyzed, and the monitoring device reports risks in surgery, including warnings, recommendations, delayed healing, increased wound infection, kidney issues, heart problems, lung issues, neurological complications, or stroke.

### FIELD OF THE DISCLOSURE

The present disclosure is generally related to a method for improved surgical care with real-time devices.

### BACKGROUND

Currently, medical monitoring devices cannot collect and inform medical professionals about a patient's glucose levels in real-time. Also, medical monitoring devices cannot analyze a patient's physiological parameters, including glucose levels, to determine recommendations to inform medical professionals. Lastly, medical monitoring devices cannot provide actionable recommendations from analyzing a patient's glucose levels with other physiological parameters. Thus, there is a need in the prior art to provide improved surgical care with real-time devices.

### DESCRIPTIONS OF THE DRAWINGS

FIG. 1: Illustrates a system for improved surgical care with real-time devices, according to an embodiment.

FIG. 2: Illustrates a method implemented by a Base Module, according to an embodiment.

FIG. 3: Illustrates a method implemented by a Glucose Module, according to an embodiment.

FIG. 4: Illustrates a method implemented by a Data Collection Module, according to an embodiment.

FIG. 5: Illustrates a method implemented by a Fuse Module, according to an embodiment.

FIG. 6: Illustrates a method implemented by a Display Module, according to an embodiment.

FIG. 7: Illustrates a Parameter Database, according to an embodiment.

FIG. 8: Illustrates a Rule Database, according to an embodiment.

FIG. 9: Illustrates a method implemented by an Integration Module, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

U.S. Pat. Nos. 10,548,503, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, are each individually incorporated herein by reference in its entirety.

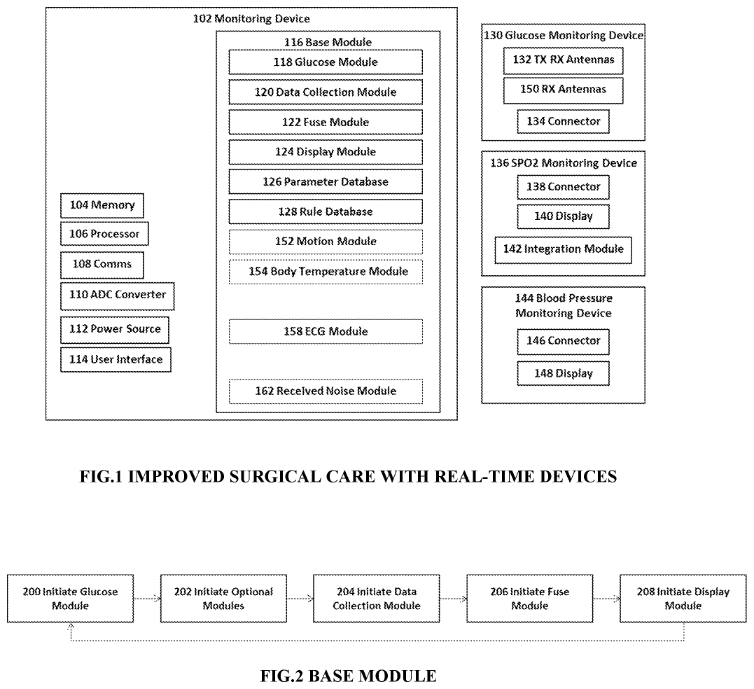

FIG. 1 illustrates a system for improved surgical care with real-time devices. The surgical care can include a surgical procedure. Surgical procedures can include pre-operative preparation, the performance of the surgical operation itself, and post-operative activities such as suturing, recovery from anesthesia, disinfection of the patient, and the like. This system comprises a monitoring device 102 that may be used to measure, detect, and display the physiological parameters of a patient to inform medical professionals of the physiological parameters. The monitoring device 102 may receive data from a plurality of connectors 134, 138, 146 to display the data collected from the glucose monitoring device 130, the SPO2 monitoring device 136, and the blood pressure monitoring device 144. The monitoring device 102 may include a memory 104, a processor 106, comms 108 such as physical connections to the plurality of devices, ADC converter 110, a power source 112, a user interface 114, and a base module 116 that may be continuously running and initiating the glucose module 118, the data collection module 120, the fuse module 122 and the display module 124. The base module 116 may optionally run one or more of the motion module 152, the body temperature module 154, the ECG module 158, and/or the received noise module 162. The monitoring device 102 may store, extract, and compare data from the parameters database 126 and the rules database 128.

Further, embodiments may include a memory 104 that may be configured to store transmitted activated RF signals by the one or more TX antennas 132 and receive a reflected portion of the transmitted activated RF signals from the one or more RX antennas 150. Further, the memory 104 may also store the converted digital processor readable format by the ADC converter 110. In one embodiment, the memory 104 may include suitable logic, circuitry, and/or interfaces that may be configured to store a machine code and/or a computer program with at least one code section executable by the processor 106. Examples of implementation of the memory 104 may include, but are not limited to, Random Access Memory (RAM), Read Only Memory (ROM), Hard Disk Drive (HDD), and/or a Secure Digital (SD) card.

Further, embodiments may include the processor 106, which may facilitate the operation of the monitoring device 102 to perform functions according to the instructions stored in the memory 104. In one embodiment, the processor 106 may include suitable logic, circuitry, interfaces, and/or code that may be configured to execute a set of instructions stored in the memory 104. The processor 106 may be configured to run the instructions obtained by the monitoring device base module 116 to perform various functions. The processor 106 may be further configured to collect real-time signals from the TX antennas 132 and the RX antennas 150 and may store the real-time signals in the memory 104. In one embodiment, the real-time signals may be assigned as initial and updated radio frequency (RF) signals. Examples of the processor 106 may be an X86-based processor, a Reduced Instruction Set Computing (RISC) processor, an Application-Specific Integrated Circuit (ASIC) processor, a Complex Instruction Set Computing (CISC) processor, and/or other processors. The processor 106 may be a multicore microcontroller specifically designed to carry multiple operations based upon pre-defined algorithm patterns to achieve the desired result. Further, the processor 106 may take inputs from the device 102 and retain control by sending signals to different parts of the glucose monitoring device 130. The processor 106 may comprise a Random Access Memory (RAM) that stores data and other results created when the processor 106 is at work. It can be noted that the data is stored temporarily for further processing, such as filtering, correlation, correction, and adjustment. Moreover, the processor 106 carries out special tasks as programs that are pre-stored in the Read Only Memory (ROM). It can be noted that the special tasks carried out by the processor 106 indicate and apply certain actions which trigger specific responses.

Further, the communication module 108 of the device 102 may communicate with the glucose monitoring device 130, SPO2 monitoring device 136, and blood pressure monitoring device 144 through the plurality of connectors 134, 138, 146. Examples of the communication module 108 may include but are not limited to, a physical connection, the Internet, a cloud network, a Wireless Fidelity (Wi-Fi) network, a Wireless Local Area Network (WLAN), a Local Area Network (LAN), Long Term Evolution (LTE), and/or a Metropolitan Area Network (MAN). In one embodiment, various devices may be configured to have a communication module integrated over circuitry arrangement to connect with the device network via various wired and wireless communication protocols, such as the cloud network. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols.

Further, embodiments may include the ADC Converter 110 which may be coupled to the RX antennas 150. The RX antennas 150 may be configured to receive the reflected activated RF signals. The ADC 110 may be configured to convert the activated RF signals from an analog signal into a digital processor readable format.

Further, embodiments may include the power source 112 which is a source of electrical energy, such as a battery or line power. Further, embodiments may include a user interface 114, which either accepts users' inputs, provides outputs to the users, or performs both actions. In one case, users can interact with the interface(s) 114 using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or a combination of the above. Further, the interface(s) may either be implemented as a Command Line Interface (CLI), a Graphical User Interface (GUI), a voice interface, or a web-based user interface.

Further, embodiments may include the base module 116 which initiates the glucose module 118, the data collection module 120, the fuse module 122, and the display module 124. Further, embodiments may include the glucose module 118, which begins by being initiated by the base module 116. The glucose module 118 sends the RF signal to transmit to the glucose monitoring device 130 TX antenna 132. For example, the TX antennas 132 may be configured to transmit the activated RF signals at a pre-defined frequency. In one embodiment, the pre-defined frequency may correspond to a range suitable for the human body. For example, the TX antennas 132 transmit activated RF signals at 122-126 GHz. The glucose module 118 stores the RF transmit signal to memory 104. The glucose module 118 receives the RF signal from the glucose monitoring device 130 RX antenna 150. For example, the RX antennas 150 may be configured to receive the reflected portion of the activated RF signals. In one embodiment, the activated RF signals may be transmitted under the user's skin, and electromagnetic energy may be reflected from many parts, such as fibrous tissue, muscle, tendons, bones, and the skin.

It can be noted that effective monitoring of the blood glucose level is facilitated by an electrical response of blood molecules, such as pancreatic endocrine hormones, against the transmitted activated RF signals. It will be apparent to a skilled person that the pancreatic endocrine hormones such as insulin and glucagon are responsible for maintaining sugar or glucose level. Further, the electromagnetic energy responded from the blood molecules may be received by the RX antennas 150. The glucose module 118 converts the received signals to digital using the ADC converter 110. The glucose module 118 stores the converted signal data in memory 104. In one embodiment, the glucose module 118 correlates the RF signals with ground truth data to determine the glucose numbers. In another embodiment, where a sensor is used to supply the correlated data, then the glucose module 118 correlates the RF signals with ground truth data to determine the glucose numbers is not needed The glucose module 118 stores the glucose number in the parameter database 126. The glucose module 118 then returns to the base module 116.

Further, embodiments may include the data collection module 120, which begins its operation by being initiated by the base module 116. The data collection module 120 receives the SPO2 data from the SPO2 monitoring device 136. The data collection module 120 stores the SPO2 data in the parameter database 126. The data collection module 120 receives the blood pressure data from the blood pressure monitoring device 144. The data collection module 120 stores the blood pressure data in the parameter database 126. The data collection module 120 then returns to the base module 116.

Further, embodiments may include the fuse module 122, which begins its operation by being initiated by the base module 116. The fuse module 122 extracts the data entry from the parameter database 126. The fuse module 122 compares the extracted data entry from the parameter database 126 to the rules database 128. The fuse module 122 determines if there is a recommendation. If it is determined that there is a recommendation, the fuse module 122 extracts the recommendation from the rules database 128. The fuse module 122 sends the extracted recommendation from the rules database 128 to the display module 124. If it is determined that there is no recommendation or after the recommendation has been sent to the display module 124, the fuse module 122 returns to the base module 116.

Further, embodiments may include the display module 124, which begins its operation by being initiated by the base module 116. The display module 124 extracts the data entry from the parameter database 126. The display module 124 sends the extracted data to the integration module 142. The display module 124 displays the data on the user interface 114. The display module 124 determines if a recommendation was received from the fuse module 122. If it is determined that a recommendation was received from the fuse module 122, the display module 124 receives it from the fuse module 122. The display module 124 displays the received recommendation on the user interface 114. If it is determined that no recommendation was received from the fuse module 122 or after the recommendation is displayed on the user interface 114, the display module 124 returns to the base module 116.

Further, embodiments may include the parameter database 126 which includes data from the data collection module 120, which collects data from the glucose monitoring device 130, SPO2 monitoring device 136, and the blood pressure monitoring device 144. The data is compared to the rule database 128 to determine if there is a relationship between the data that requires the medical professional's attention and provides the medical professional with a notification. The data is extracted from the parameter database 126 and displayed on the monitoring device 102 user interface 114 to inform the medical professional of the patient's physiological parameters. The database contains a patient ID, the time in which the data was collected and displayed on the monitoring device 102, the patient's glucose numbers, oxygen saturation levels, and blood pressure. In some embodiments, the database may include temperature, heart rate, alertness, activity, etc.

Further, embodiments may include the rule database 128, which contains a predetermined set of rules that provide a medical professional with a notification or alert on the monitoring device 102 if the patient's physiological parameters are at certain levels or exceed certain thresholds that may cause harm to the patient. The rule database 128 is used with the fuse module 122 in which the data from the parameter database 126 is compared to the thresholds provided in the rule database 128 to determine if the patient's physiological parameters exceed one, two, or a combination of the thresholds and, if so, the rule or recommendation is extracted and is displayed on the monitoring device 102 user interface 114. The database may contain thresholds for the patient's glucose numbers, oxygen saturation levels, blood pressure, and the corresponding rule. In some embodiments, the database may contain thresholds for the patient's temperature, heart rate, alertness, activity, etc.

Further, the glucose monitoring device 130 may be connected to the monitoring device 102 through the connector 134. In one embodiment, the connector 134 may be a wireless and/or wired communication channel. The glucose monitoring device 130 may be worn by the user. The glucose monitoring device 130 may determine health parameters using activated RF signals. In one embodiment, the health parameters may include blood sugar or blood glucose levels. The system may target specific blood vessels using the activated RF signals, which may output signals, and the output signals may correspond to the blood glucose level in the user. In one embodiment, the system may include integrated circuit (IC) devices (not shown) with transmit and/or receive antennas integrated therein.

Monitoring the blood glucose level of the user using the activated RF signals involves the transmission of suitable activated RF signals below the user's skin surface. Corresponding to the transmission, a reflected portion of the activated RF signals is received on multiple receive antennas. Further, the system isolates and/or processes a signal in response to the received activated RF signals. The system may output a signal from the received activated RF signals that correspond to the blood glucose level in the user. It can be noted that the glucose monitoring device 130 may be worn by the user at various locations such as wrist, arm, leg, etc. In one embodiment, the system for monitoring the blood glucose level of the user using the activated RF signals involves transmitting activated RF signals below the skin surface, receiving a reflected portion of the activated RF signals on multiple receive antennas, isolating a signal from the activated RF signals at a particular location in response to the received activated RF signals and outputting a signal that corresponds to the blood glucose level in the user in response to the isolated signal. In one embodiment, beamforming is used in the receiving process to isolate the activated RF signals reflected from a specific location on a specific blood vessel to provide a high-quality signal corresponding to the blood glucose levels in the specific blood vessel. In another embodiment, Doppler effect processing may be used to isolate the activated RF signals reflected from the specific blood vessel's specific location to provide the high-quality signal corresponding to the blood glucose levels in the specific blood vessel. It can be noted that analog and/or digital signal processing techniques may be used to implement beamforming and/or Doppler effect processing and digital signal processing of the received signals to dynamically adjust a received beam onto the desired location. In another embodiment, the beamforming and the Doppler effect processing may be used together to isolate the activated RF signals reflected from the specific location in the specific blood vessel to provide the high-quality signal corresponding to the blood glucose levels in the specific blood vessel.

In one exemplary embodiment, activated RF signals of a frequency range of 122-126 gigahertz (GHz) having a shallower penetration depth are used to monitor blood glucose levels. It can be noted that the shallower penetration depth reduces undesirable reflections, such as reflections from bone, and dense tissue, such as tendons, ligaments, and muscle, which may reduce the signal processing burden and improve the quality of the desired signal that reflects from the location of the blood vessel. It can also be noted that bones are dielectric and semi-conductive. In addition, bones are anisotropic, so not only are bones conductive, but they also conduct differently depending on the direction of the flow of current through the bone. Alternatively, the bones are also piezoelectric materials. Therefore, activated RF signals of a frequency range of 122-126 GHZ with the shallower penetration depth may be used to monitor blood glucose levels.

Further, the glucose monitoring device 130 may comprise the one or more transmission (TX) antennas 132, the one or more receiving (RX) antennas 132, and the connector 134. In one embodiment, the glucose monitoring device 130 may be wearable and portable such as, but not limited to, a cell phone, a smartwatch, a tracker, a wearable monitor, a wristband, and a personal blood monitoring device. The TX antennas 132 and the RX antennas 150 may be fabricated on a substrate (not shown) within the glucose monitoring device 130 in a suitable configuration. In one exemplary embodiment, at least two TX antennas 132 and at least four RX antennas 150 are fabricated on the substrate. The TX antennas 132 and the RX antennas 150 may correspond to a circuitry arrangement (not shown) on the substrate. In some embodiments, the glucose monitoring device 130 may communicate with the monitoring device 102 wirelessly to connect to the monitoring device 102 user interface 114. Further, embodiments may include a plurality of TX antennas 132 and RX antennas 150. The TX antennas 132 and the RX antennas 150 may be integrated into the circuitry arrangement. The TX antennas 132 may be configured to transmit the activated RF signals at a pre-defined frequency. In one embodiment, the pre-defined frequency may correspond to a range suitable for the human body. For example, the TX antennas 132 transmit activated RF signals at 122-126 GHz. Successively, the RX antennas 150 may be configured to receive the reflected portion of the activated RF signals. In one embodiment, the activated RF signals may be transmitted beneath the user's skin, and electromagnetic energy may be reflected from many parts, such as fibrous tissue, muscle, tendons, bones, and the skin.

It can be noted that effective monitoring of the blood glucose level is facilitated by an electrical response of blood molecules, such as pancreatic endocrine hormones, against the transmitted activated RF signals. It will be apparent to a skilled person that the pancreatic endocrine hormones such as insulin and glucagon are responsible for maintaining sugar or glucose level. Further, the electromagnetic energy reflected from the blood molecules may be received by the RX antennas 150.

Further, embodiments may include the connector 134, such as a data cable designed for data transfer between the glucose monitoring device 130 and the monitoring device 102 to send the glucose data to the monitoring device 102 to be displayed on the user interface 114. For example, the connector 134 may transmit electronic information from the glucose monitoring device 130 to the monitoring device 102 and, in some embodiments, the monitoring device 102 to the glucose monitoring device 130.

Further, embodiments may include the SPO2 monitoring device 136 which measures a person's oxygen saturation levels through a noninvasive pulse oximetry method. For example, the most common approach is transmissive pulse oximetry, where a sensor device is placed on a thin part of the patient's body, usually a fingertip, earlobe, or an infant's foot. Fingertips and earlobes have higher blood flow rates than other tissues, facilitating heat transfer. The device passes two wavelengths of light through the body part to a photodetector. It measures the changing absorbance at each wavelength, allowing it to determine the absorbances due to the pulsing arterial blood alone, excluding venous blood, skin, bone, muscle, fat, and, in most cases, nail polish. For example, oxygen saturation is the fraction of oxygen-saturated hemoglobin relative to total hemoglobin (unsaturated+saturated) in the blood. The human body requires and regulates a very precise and specific balance of oxygen in the blood. Normal arterial blood oxygen saturation levels in humans are 97-100 percent. It is considered low and hypoxemia if the level is below 90 percent. Arterial blood oxygen levels below 80 percent may compromise organ function, such as the brain and heart, and should be promptly addressed. Continued low oxygen levels may lead to respiratory or cardiac arrest.

In some embodiments, the SPO2 monitoring device 136 may receive data from the monitoring device 102 through the integration module 142 to integrate glucose data with the oxygen saturation data to determine a patient's underlying conditions, such as the risk of diabetes or cardiovascular issues.

Further, embodiments may include the connector 138, such as a data cable designed for data transfer between the SPO2 monitoring device 136 and the monitoring device 102 to send the oxygen saturation data to the monitoring device 102 to be displayed on the user interface 114. For example, the connector 138 may transmit electronic information from the SPO2 monitoring device 136 to the monitoring device 102 and, in some embodiments, the monitoring device 102 to the SPO2 monitoring device 136.

Further, embodiments may include the display 140, which may either accept users' inputs, provide outputs to the users, or perform both actions. In one case, a user can interact with the display 140 using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or a combination of the above. Further, the display 140 may be implemented as a Command Line Interface (CLI), a Graphical User Interface (GUI), a voice interface, or a web-based user interface.

Further, embodiments may include the integration module 142, which begins its operation by continuously polling to receive the data from the display module 124. The integration module 142 receives the data from the display module 124. The integration module 142 integrates the received data into the SPO2 monitoring device 136. The integration module 142 displays the integrated data on the SPO2 monitoring device 136.

In an embodiment, the SPO2 monitoring device 136 may have a construction that is similar to the glucose monitoring device 130 including having one or more TX antennas and one or more RX antennas so as to use the transmission of RF activated signals by the transmit antenna(s) and receipt of returning RF signals by the receive antenna(s) to determine the SPO2 level of the patient.

In an embodiment, the glucose monitoring device 130 can be used to also detect the SPO2 level of the patient.

Further, embodiments may include the blood pressure monitoring device 144 which may be a device used to measure blood pressure, for example, a device composed of an inflatable cuff to collapse and then release the artery under the cuff in a controlled manner, and a mercury or aneroid manometer to measure the pressure. For example, a sphygmomanometer consists of an inflatable cuff, a measuring unit, such as the mercury manometer or aneroid gauge, and a mechanism for inflation, which may be a manually operated bulb and valve or a pump operated electrically. For example, digital blood pressure meters employ oscillometric measurements and electronic calculations rather than auscultation. They measure systolic and diastolic pressures by oscillometric detection, employing either deformable membranes that are measured using differential capacitance or piezoresistance, and they include a microprocessor.

In an embodiment, the blood pressure monitoring device 144 may have a construction that is similar to the glucose monitoring device 130 including having one or more TX antennas and one or more RX antennas so as to use the transmission of RF activated signals by the transmit antenna(s) and receipt of returning RF signals by the receive antenna(s) to determine the blood pressure level of the patient.

In an embodiment, the glucose monitoring device 130 can be used to also detect the blood pressure level of the patient.

Further, embodiments may include the connector 146, such as a data cable designed for data transfer between the blood pressure monitoring device 144 and the monitoring device 102 to send the blood pressure data to the monitoring device 102 to be displayed on the user interface 114. For example, the connector 146 may transmit electronic information from the blood pressure monitoring device 144 to the monitoring device 102 and, in some embodiments, data may be sent from the monitoring device 102 to the blood pressure monitoring device 144.

Further, embodiments may include the display 148, which may either accept users' inputs, provide outputs to the users, or perform both actions. In one case, a user can interact with the display 148 using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or a combination of the above. Further, the display 148 may be implemented as a Command Line Interface (CLI), a Graphical User Interface (GUI), a voice interface, or a web-based user interface.

FIG. 2 illustrates an example operation of the base module 116. The operation begins with the base module 116 initiating, at step 200, the glucose module 118. For example, the glucose module 118 begins by being initiated by the base module 116. The glucose module 118 sends the RF signal to transmit to the glucose monitoring device 130 TX antenna 132. For example, the TX antenna 132 may be configured to transmit the activated RF signals at a pre-defined frequency. In one embodiment, the pre-defined frequency may correspond to a range suitable for the human body. For example, the TX antenna 132 transmits activated RF signals at 122-126 GHz. The glucose module 118 stores the RF transmit signal to memory 104. The glucose module 118 receives the RF signal from the glucose monitoring device 130 RX antenna 150. For example, the RX antenna 150 may be configured to receive the reflected portion of the activated RF signals. The glucose module 118 converts the received signal to digital using the ADC converter 110. The glucose module 118 stores the converted signal data in memory 104. The glucose module 118 correlates the RF signals with ground truth data to determine the glucose numbers. The glucose module 118 stores the glucose number in the parameter database 126. The glucose module 118 then returns to the base module 116.

The base module 116 may initiate one or more optional modules at step 202 to improve the accuracy of the glucose module 118. In some embodiments, the base module 116 may utilize the motion module 152 that includes at least one sensor from the group of an accelerometer, a gyroscope, an inertial movement sensor, or another similar sensor. The motion module 152 may have its own processor or utilize the processor 120 to calculate the user's movement. Motion from the user will change the blood volume in a given portion of their body and the blood flow rate in their circulatory system. This may cause noise, artifacts, or other errors in the real-time signals received by the RX antennas 150. The motion module 152 may compare the calculated motion to a threshold stored in memory 104. For example, the motion threshold could be movement of more than two centimeters in one second. The motion threshold could be near zero to ensure the user is stationary when measuring to ensure the least noise in the RF signal data. When calculated motion levels exceed the threshold, the motion module 152 may flag the RF signals collected at the time stamp corresponding to the motion as potentially inaccurate. In some embodiments, the motion module 152 may compare RF signal data to motion data over time to improve the accuracy of the motion threshold. The motion module 152 may alert the nurse, doctor, or medical staff, such as with an audible beep, warning, text message, or alert to a connected mobile device. The alert would signal the nurse, doctor, or medical that the patient is moving too much to get an accurate measurement. The motion module 152 may update the parameter database 126 with the calculated motion of the user that corresponds with the received RF signal data. In this manner, the motion module 152 may be simplified to just collect motion data and allow the base module 116 to determine if the amount of motion calculated exceeds a threshold that would indicate the received RF signal data is too noisy to be relied upon for a blood glucose measurement.

The base module 116 may optionally utilize the body temperature module 154 that includes at least one sensor from the group of a thermometer, a platinum resistance thermometer (PRT), a thermistor, a thermocouple, or another temperature sensor. The body temperature module 154 may have its own processor or utilize the processor 120 to calculate the temperature of the user or the user's environment. The user's body temperature, the environmental temperature, and the difference between the two will change the blood volume in a given part of their body and the blood flow rate in their circulatory system. Variations in temperature from the normal body or room temperature may cause noise, artifacts, or other errors in the real-time signals received by the RX antennas 150. The body temperature module 154 may compare the measured temperature to a threshold temperature stored in memory 104. For example, the environmental temperature threshold may be set at zero degrees Celsius because low temperatures can cause a temporary narrowing of blood vessels which may increase the user's blood pressure. When the measured temperature exceeds the threshold, the body temperature module 154 may flag the RF signals collected at the time stamp corresponding to the temperature as potentially being inaccurate. In some embodiments, the body temperature module 154 may compare RF signal data to temperature data over time to improve the accuracy of the temperature threshold. The body temperature module 154 may alert the nurse, doctor, or medical staff, such as with an audible beep or warning or a text message or alert to a connected mobile device. The alert would signal to the nurse, doctor, or medical staff that their body or environmental temperature is not conducive to getting an accurate measurement. The body temperature module 154 updates the parameter database 126 with the measured user or environmental temperature corresponding to the received RF signal data. In this manner, the body temperature module 154 may be simplified to just collect temperature data and allow the base module 116 to determine if the temperature measure exceeds a threshold that would indicate the received RF signal data is too noisy to be relied upon for a blood glucose measurement.

The base module 116 may optionally utilize the ECG module 158 that includes at least one electrocardiogram sensor. The ECG module 158 may have its own processor or utilize the processor 120 to record the electrical signals that correspond with the user's heartbeat. The user's heartbeat will impact blood flow. Measuring the ECG data may allow the received RF data to be associated with peak and minimum cardiac output to create a pulse waveform allowing for the estimation of blood volume at a given point in the wave of ECG data. Variations in blood volume may cause noise, artifacts, or other errors in the real-time signals received by the RX antennas 150. The ECG module 158 may compare the measured cardiac data to a threshold stored in memory 104. For example, the threshold may be a pulse above 160 bpm, as the increased blood flow volume may cause too much noise in the received RF signal data to accurately measure the blood glucose. When the ECG data exceeds the threshold, the ECG module 158 may flag the RF signals collected at the time stamp corresponding to the ECG data as potentially being inaccurate. In some embodiments, the ECG module 158 may compare RF signal data to ECG data over time to improve the accuracy of the ECG data threshold or to improve the measurement of glucose at a given point in the cycle between peak and minimum cardiac output. The ECG module 158 may alert the nurse, doctor, or medical staff, such as with an audible beep, warning, text message, or alert to a connected mobile device. The alert would signal to the nurse, doctor, or medical staff that the patient's heart rate is not conducive to getting an accurate measurement or requires additional medical intervention. The ECG module 158 may update the parameter database 126 with the measured ECG data that corresponds with the received RF signal data. In this manner, the ECG module 158 may be simplified to just collect ECG data and allow the base module 116 to determine if the ECG data exceeded a threshold that would indicate the received RF signal data is too noisy to be relied upon for a blood glucose measurement.

The base module 116 may optionally utilize the received noise module 162 that includes at least one sensor measuring background signals such as RF signals, Wi-Fi, and other electromagnetic signals that could interfere with the signals received by the RX antennas 150. The received noise module 162 may have its own processor or utilize the processor 120 to calculate the level of background noise received. Background noise may interfere with or cause noise, artifacts, or other errors or inaccuracies in the real-time signals received by the RX antennas 150. The received noise module 162 may compare the level and type of background noise to a threshold stored in memory 104. The threshold may be in terms of field strength (volts per meter and ampere per meter) or power density (watts per square meter). For example, the threshold may be RF radiation greater than 300 μW/m². When the background noise data exceeds the threshold, the received noise module 162 may flag the RF signals collected at the time stamp corresponding to background noise levels as potentially inaccurate. In some embodiments, the received noise module 162 may compare RF signal data to background noise over time to improve the accuracy of the noise thresholds. The received radiation module may alert the nurse, doctor, or medical staff, such as with an audible beep or warning, a text message, or an alert to a connected mobile device. The alert would signal to the nurse, doctor, or medical staff that the current background noise level is not conducive to getting an accurate measurement. The received noise module 162 may update the parameter database 126 with the background noise data corresponding to the received RF signal data. In this manner, the received noise module 162 may be simplified to just collect background noise data and allow the base module 116 to determine if the measure exceeded a threshold that would indicate the received RF signal data is too noisy to be relied upon for a blood glucose measurement, or if an alternative transfer function should be used to compensate for the noise.

The base module 116 initiates, at step 204, the data collection module 120. For example, the data collection module 120 begins by being initiated by the base module 116. The data collection module 120 receives the SPO2 data from the SPO2 monitoring device 136. The data collection module 120 stores the SPO2 data in the parameter database 126. The data collection module 120 receives the blood pressure data from the blood pressure monitoring device 144. The data collection module 120 stores the blood pressure data in the parameter database 126. The data collection module 120 then returns to the base module 116.

The base module 116 initiates, at step 206, the fuse module 122. For example, the fuse module 122 begins by being initiated by the base module 116. The fuse module 122 extracts the data entry from the parameter database 126. The fuse module 122 compares the extracted data entry from the parameter database 126 to the rules database 128. The fuse module 122 determines if there is a recommendation. If it is determined that there is a recommendation, the fuse module 122 extracts the recommendation from the rules database 128. The fuse module 122 sends the extracted recommendation from the rules database 128 to the display module 124. If it is determined that there is no recommendation or after the recommendation has been sent to the display module 124, the fuse module 122 returns to the base module 116.

The base module 116 initiates, at step 208, the display module 124. For example, the display module 124 begins by being initiated by the base module 116. The display module 124 extracts the data entry from the parameter database 126. The display module 124 displays the data on the user interface 114. The display module 124 determines if a recommendation was received from the fuse module 122. If it is determined that a recommendation was received from the fuse module 122, the display module 124 receives it from the fuse module 122. The display module 124 displays the received recommendation on the user interface 114. If it is determined that no recommendation was received from the fuse module 122 or after the recommendation is displayed on the user interface 114, the display module 124 returns to the base module 116.

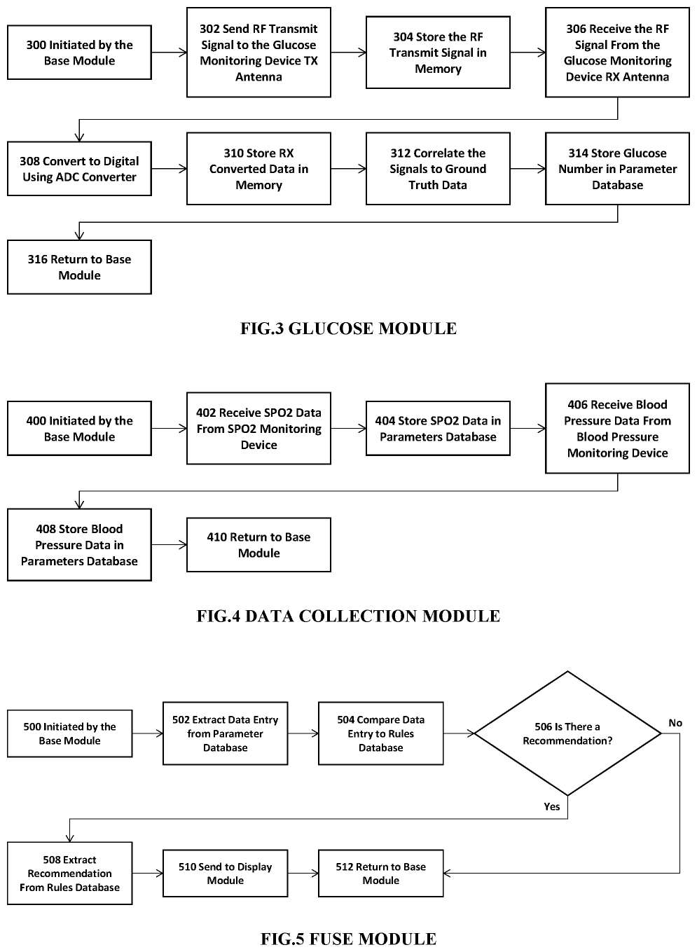

FIG. 3 illustrates an example operation of the glucose module 118. The operation begins with the glucose module 118 being initiated, at step 300, by the base module 116. The glucose module 118 sends, at step 302, the RF signal to transmit to the glucose monitoring device 130 TX antenna 132. For example, the TX antenna 132 may be configured to transmit the activated RF signals at a pre-defined frequency. In one embodiment, the pre-defined frequency may correspond to a range suitable for the human body. For example, the TX antenna 132 transmits activated RF signals at 122-126 GHz. The glucose module 118 stores, at step 304, the RF signal to transmit to memory 104. For example, the glucose module 118 stores the transmitted signal to memory 104, such as activated RF signals at 122-126 GHz. The glucose module 118 receives, at step 306, the RF signal from the glucose monitoring device 130 RX antenna 150. For example, the RX antenna 150 may be configured to receive the reflected portion of the activated RF signals. The glucose module 118 converts, at step 308, to digital using the ADC converter 110. For example, the ADC converter 110 may be configured to convert the activated RF signals from an analog signal into a digital processor readable format. The glucose module 118 stores, at step 310, the converted signal data in memory 104. For example, the glucose module 118 stores the received signal from the glucose monitoring device 130 RX antenna 150 that has been converted to a digital processor readable format in memory 104. The glucose module 118 correlates, at step 312, the RF signal with ground truth data to determine the glucose number. For example, the glucose module 118 may be configured to execute an AI correlation between the real-time ground truth data and the converted data at step 308. In one embodiment, the AI correlation between the real-time ground truth data and the converted data is executed to determine whether the converted data corresponds to the real-time ground truth data. For example, the glucose module 118 executes the AI correlation between the real-time ground truth data related to the blood glucose level of the patient as 110 mg/dL corresponding to the radio signal of frequency 122 GHZ, and the 8-bit data corresponding to activated RF signal of frequency range 140-155 GHz. The memory 104 may store the real-time ground truth data and converted data. For example, the memory 104 stores the real-time ground truth data related to the patient's blood glucose level as 110 mg/dL corresponding to the radio signal of frequency 122 GHz, and the 8-bit data corresponding to activated RF signal of frequency range 140-155 GHz. The glucose module 118 stores, at step 314, the glucose number in the parameter database 126. For example, the glucose module 118 stores the patient's glucose levels in the parameter database 126, such as 220 mg/dL, 219 mg/dL, etc. The glucose module 118 then returns, at step 316, to the base module 116.

FIG. 4 illustrates an example operation of the data collection module 120. The operation begins with the data collection module 120 being initiated, at step 400, by the base module 116. For example, the data collection module 120 may be initiated once the glucose module 118 determines the patient's glucose numbers. In some embodiments, the data collection module 120 may be continuously running to collect the patient's oxygen saturation levels and blood pressure from the SPO2 monitoring device 136 and the blood pressure monitoring device 144. The data collection module 120 receives, at step 402, the SPO2 data from the SPO2 monitoring device 136. For example, the data collection module 120 receives the patient's oxygen saturation levels from the SPO2 monitoring device 136. For example, the SPO2 monitoring device 136 may measure a person's oxygen saturation levels through a noninvasive pulse oximetry method. For example, the most common approach is transmissive pulse oximetry, where a sensor device is placed on a thin part of the patient's body, usually a fingertip, earlobe, or an infant's foot. Fingertips and earlobes have higher blood flow rates than other tissues, facilitating heat transfer. The device passes two wavelengths of light through the body part to a photodetector. It measures the changing absorbance at each wavelength, allowing it to determine the absorbances due to the pulsing arterial blood alone, excluding venous blood, skin, bone, muscle, fat, and, in most cases, nail polish. For example, oxygen saturation is the fraction of oxygen-saturated hemoglobin relative to total hemoglobin (unsaturated+saturated) in the blood. The human body requires and regulates a very precise and specific balance of oxygen in the blood. Normal arterial blood oxygen saturation levels in humans are 97-100 percent. It is considered low and hypoxemia if the level is below 90 percent. Arterial blood oxygen levels below 80 percent may compromise organ function, such as the brain and heart, and should be promptly addressed. Continued low oxygen levels may lead to respiratory or cardiac arrest. The data collection module 120 stores, at step 404, the SPO2 data in the parameter database 126. For example, the data collection module 120 stores the patient's real-time oxygen saturation levels in the parameter database 126. The data collection module 120 receives, at step 406, the blood pressure data from the blood pressure monitoring device 144. For example, the data collection module 120 receives the patient's blood pressure data from the blood pressure monitoring device 144. For example, the blood pressure monitoring device 144 may be a device used to measure blood pressure, for example, a device composed of an inflatable cuff to collapse and then releases the artery under the cuff in a controlled manner, and a mercury or aneroid manometer to measure the pressure. For example, a sphygmomanometer consists of an inflatable cuff, a measuring unit, such as the mercury manometer or aneroid gauge, and a mechanism for inflation, which may be a manually operated bulb and valve or a pump operated electrically. For example, digital meters employ oscillometric measurements and electronic calculations rather than auscultation. They measure systolic and diastolic pressures by oscillometric detection, employing either deformable membranes that are measured using differential capacitance or piezoresistance, and they include a microprocessor. The data collection module 120 stores, at step 408, the blood pressure data in the parameter database 126. For example, the data collection module 120 stores the patient's real-time blood pressure data in the parameter database 126. The data collection module 120 then returns, at step 410, to the base module 116.

FIG. 5 illustrates an example operation of the fuse module 122. The operation begins with the fuse module 122 being initiated, at step 500, by the base module 116. For example, the fuse module 122 may be initiated once the patient's real-time glucose, oxygen saturation, and blood pressure data are collected. In some embodiments, the fuse module 122 may be initiated once a new data entry is added to the parameter database 126 by continuously querying the parameter database 126 for a new data entry. The fuse module 122 extracts, at step 502, the data entry from the parameter database 126. For example, the fuse module 122 extracts the new data entry from the parameter database 126, including the patient ID, the time in which the data was collected and displayed on the monitoring device 102, the patient's glucose numbers, the patient's oxygen saturation levels, and the patient's blood pressure. In some embodiments, the database may include temperature, heart rate, alertness, activity, etc. The fuse module 122 compares, at step 504, the extracted data entry from the parameter database 126 to the rules database 128. For example, the fuse module 122 may compare the patient's real-time glucose, oxygen saturation, and blood pressure data to the rules database to determine if any of the patient's physiological parameters exceed any thresholds stored in the rule database 128. In some embodiments, the patient's temperature, heart rate, alertness, activity, etc., may be compared to the rules database 128. The fuse module 122 determines, at step 506, if there is a recommendation. For example, if the patient's physiological parameters exceed any thresholds stored in the rule database 128 the corresponding rule may be to notify the medical professionals with a recommendation displayed on the monitoring device 102 user interface 114. For example, if the patient's glucose levels rise and exceed the threshold in the rule database 128 along with the patient's blood pressure decreasing past the thresholds stored in the rule database 128, the corresponding rule may be to notify the medical professional to provide the patient with insulin to lower the patient's glucose levels. For example, suppose the patient's glucose levels decrease. In that case, the oxygen saturation levels decrease, and the patient's blood pressure increases past the thresholds. The corresponding rule may be to provide the patient with dextrose. A warning may be provided to the medical professional that the patient is experiencing low blood sugar, such as hypoglycemia. For example, if the patient's oxygen saturation levels decrease and blood pressure increases, the rule may be to provide the patient with oxygen. For example, if the patient's blood pressure increases, the rule may be to give them medication to lower their blood pressure. For example, the fuse module 122 may extract a recommendation based on the patient's oxygen saturation levels, glucose numbers, and blood pressure. For example, oxygen saturation is correlated with glucose levels in which glucose and arterial pressure variability are associated with markers of obstructive sleep apnea syndrome, or OSAS, severity among nondiabetic patients with lower oxygen saturation levels. Glucose and arterial pressure variability in OSAS may be used as an indicator of future diabetes in nondiabetic patients and increased cardiovascular risk. The recommendation that would be extracted would be that the patient has an increased risk of diabetes and cardiovascular risks. If it is determined that there is a recommendation, the fuse module 122 extracts, at step 508, the recommendation from the rules database 128. For example, the fuse module 122 may extract the rule, such as to notify the medical professional to provide the patient with insulin to lower the patient's glucose levels, to provide the patient with dextrose, and a warning may be provided to the medical professional that the patient is experiencing low blood sugar, be to provide the patient with oxygen, to provide the patient with medication to lower their blood pressure, etc. The fuse module 122 sends, at step 510, the extracted recommendation from the rules database 128 to the display module 124. For example, the fuse module 122 sends the extracted rule, recommendation, notification, etc., to the display module 124. If it is determined that there is no recommendation or after the recommendation has been sent to the display module 124, the fuse module 122 returns, at step 512, to the base module 116.

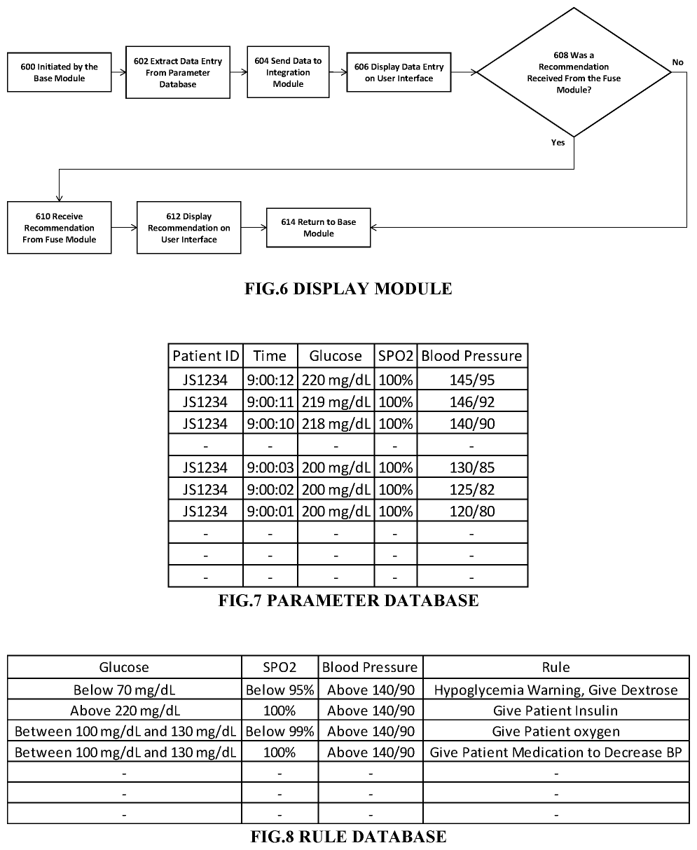

FIG. 6 illustrates an example operation of the display module 124. The operation begins with the display module 124 being initiated, at step 600, by the base module 116. For example, the display module 124 may be initiated once the fuse module 122 has been completed. In some embodiments, the display module 124 may be initiated once a new data entry is stored in the parameter database 126 to display the patient's physiological parameters. The display module 124 extracts, at step 602, the data entry from the parameter database 126. For example, the display module 124 extracts the new data entry from the parameter database 126, including the patient ID, the time in which the data was collected and displayed on the monitoring device 102, the patient's glucose numbers, the patient's oxygen saturation levels, and the patient's blood pressure. In some embodiments, the database may include temperature, heart rate, alertness, activity, etc. The display module 124 sends, at step 604, the data entry from the parameter database 126 to the integration module 142. For example, the display module 124 sends the new data entry from the parameter database 126, including the patient ID, the time in which the data was collected, the patient's glucose numbers, the patient's oxygen saturation levels, and the patient's blood pressure. In some embodiments, the data may include temperature, heart rate, alertness, activity, etc. The display module 124 displays, at step 606, the data on the user interface 114. For example, the display module 124 displays the new data entry from the parameter database 126 on the user interface 114, including the patient ID, the time in which the data was collected and displayed on the monitoring device 102, the patient's glucose numbers, the patient's oxygen saturation levels, and the patient's blood pressure. In some embodiments, the database may include temperature, heart rate, alertness, activity, etc. In some embodiments, the data may be displayed on the user interface 114 as an individual number, shown as a graph over time, etc., to inform the medical professionals of the patient's current physiological parameters. The display module 124 determines, at step 608, if a recommendation was received from the fuse module 122. For example, the display module 124 may determine if a recommendation, rule, notification, etc., was received from the fuse module 122. If it is determined that there was a recommendation received from the fuse module 122, the display module 124 receives, at step 610, the recommendation from the fuse module 122. For example, the display module 122 may receive a recommendation, rule, notification, etc., from the fuse module 122, such as to notify the medical professional to provide the patient with insulin to lower the patient's glucose levels, to provide the patient with dextrose and a warning may be provided to the medical professional that the patient is experiencing low blood sugar, be to provide the patient with oxygen, to provide the patient with medication to lower their blood pressure, etc. The display module 124 displays, at step 612, the received recommendation on the user interface 114. For example, the display module 124 displays the received recommendation, rule, notification, etc., on the user interface 114, such as to notify the medical professional to provide the patient with insulin to lower the patient's glucose levels, to provide the patient with dextrose and a warning may be provided to the medical professional that the patient is experiencing low blood sugar, be to provide the patient with oxygen, to provide the patient with medication to lower their blood pressure, etc. If it is determined that no recommendation was received from the fuse module 122 or after the recommendation is displayed on the user interface 114, the display module 124 returns, at step 614, to the base module 116.

FIG. 7 illustrates an example of the parameter database 126. The database 126 is created from the process described in the data collection module 120, which collects data from the glucose monitoring device 130, SPO2 monitoring device 136, and the blood pressure monitoring device 144. The data is compared to the rule database 128 to determine if there is a relationship between the data that requires the medical professional's attention and provides the medical professional with a notification. The data is extracted from the rules database 128 and parameters database 126 and displayed on the monitoring device 102 user interface 114 to inform the medical professional of the patient's physiological parameters. The database 126 contains a patient ID, the time in which the data was collected and displayed on the monitoring device 102, the patient's glucose numbers, oxygen saturation levels, and blood pressure. In some embodiments, the database 126 may include temperature, heart rate, alertness, activity, etc.

FIG. 8 illustrates an example of the rule database 128. The database 128 may contain a predetermined set of rules that provide a medical professional with a notification or alert on the monitoring device 102 if the patient's physiological parameters are at certain levels or exceed certain thresholds that may cause harm to the patient. The rules database 128 is used in the process described in the fuse module 122 in which the data from the parameter database 126 is compared to the thresholds provided in the rule database 128 to determine if the patient's physiological parameters exceed one, two, or a combination of the thresholds and if so, the rule or recommendation is extracted and is displayed on the monitoring device 102 user interface 114. The rules database 128 may contain thresholds for the patient's glucose numbers, oxygen saturation levels, blood pressure, and the corresponding rule. In some embodiments, the database 128 may contain thresholds for the patient's temperature, heart rate, alertness, activity, etc.

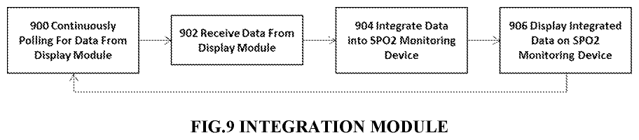

FIG. 9 illustrates an example operation of the integration module 142. The operation begins with the integration module 142 continuously polling at step 900 to receive the data from the display module 124. For example, the integration module 142 is continuously polling to receive the data from the display module 124, such as the patient ID, the time in which the data was collected, the patient's glucose numbers, and the patient's blood pressure. In some embodiments, the data may include temperature, heart rate, alertness, activity, etc. The integration module 142 receives, at step 902, the data from the display module 124. For example, the integration module 142 receives the data from the display module 124, such as the patient ID, the time in which the data was collected, the patient's glucose numbers, and the patient's blood pressure. In some embodiments, the data may include temperature, heart rate, alertness, activity, etc. The integration module 142 integrates, at step 904, the received data into the SPO2 monitoring device 136. For example, the integration module 142 may integrate the received data with the oxygen saturation data collected by the SPO2 monitoring device 136, such as the received glucose numbers, blood pressure, temperature, heart rate, alertness, activity, etc. For example, oxygen saturation is correlated with glucose levels in which glucose and arterial pressure variability are associated with markers of obstructive sleep apnea syndrome, or OSAS, severity among nondiabetic patients with lower oxygen saturation levels. Glucose and arterial pressure variability in OSAS may be used as an indicator of future diabetes in nondiabetic patients and increased cardiovascular risk. The integration module 142 displays, at step 906, the integrated data on the SPO2 monitoring device 136. For example, the integration module 142 displays the integrated data on the SPO2 monitoring device 136, such as the oxygen saturation levels, glucose numbers, blood pressure, temperature, heart rate, alertness, activity, increased risk for diabetes or cardiovascular risks, etc.

Functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A method of health monitoring in a patient during a surgical procedure, the method comprising:
during the surgical procedure on the patient, non-invasively measuring a glucose level of the patient during the surgical procedure using a sensor, wherein non-invasively measuring the glucose level of the patient during the surgical procedure includes:
transmitting, using one or more transmit antennas of the sensor, one or more transmit signals into the patient during the surgical procedure, wherein the one or more transmit signal are radio frequency signals;
receiving, using one or more receive antennas of the sensor, one or more response signals responsive to the one or more transmit signals from the patient during the surgical procedure, wherein the one or more response signals are radio frequency signals;
converting the one or more response signals to one or more digital response signals using an analog-to-digital converter of the sensor; and
processing the one or more digital response signals to determine the glucose level of the patient during the surgical procedure;
during the surgical procedure on the patient, measuring an oxygen saturation level of the patient using an SPO2 monitoring device;
storing the measured glucose level of the patient in a time entry of a parameter database that is separate from and in communication with the sensor;
storing the measured oxygen saturation level in the time entry of the parameter database;
extracting the measured glucose level from the parameter database and using the measured glucose level in a comparison with a glucose range in a data entry of a feedback database that is separate from the sensor, the feedback database includes a plurality of separate data entries, each separate data entry of the plurality of separate data entries includes a predetermined rule, a corresponding glucose range, a corresponding oxygen saturation range that corresponds to the predetermined rule, and a complication risk;
extracting the measured oxygen saturation level from the parameter database and using the measured oxygen saturation level in a comparison with the corresponding oxygen saturation range in the data entry of the feedback database;
extracting the predetermined rule of the data entry of the feedback database for display if the measured glucose level is within the corresponding glucose range of the data entry and if the measured oxygen saturation level is within the corresponding oxygen saturation range of the data entry, wherein the predetermined rule includes directing administration of one or more of insulin, dextrose, oxygen, or blood pressure medication to the patient during the surgical procedure;
extracting the complication risk of the data entry of the feedback database for display if the measured glucose level is within the corresponding glucose range of the data entry and if the measured oxygen saturation level is within the corresponding oxygen saturation range of the data entry; and
displaying the predetermined rule, the measured glucose level, the measured oxygen saturation level, and the complication risk on the SPO2 monitoring device.

2-4. (canceled)

5. The method of claim 1, wherein the measuring of the glucose level is performed in real-time during the surgical procedure.

6. The method of claim 1, wherein the measuring of the glucose level is performed continuously during an entire duration of the surgical procedure.

7. The method of claim 1, wherein the measuring of the oxygen saturation level is performed continuously during an entire duration of the surgical procedure.

8. The method of claim 1, wherein the complication risk includes a risk of one or more of: wound infection, kidney complications, heart complications, lung complications, neurological complications, and/or stroke.

9. (canceled)

10. The method of claim 1, further comprising combining the measured glucose level and the measured oxygen saturation level based on times of measurement.

11-13. (canceled)

14. The method of claim 1, wherein the complication risk includes a diagnosis of obstructive sleep apnea syndrome.

15-22. (canceled)
